(19) United States
(12) Patent Application Publication
(10) Pub. No.: US 2023/0276829 A1
(43) Pub. Date: Sep. 7, 2023

(54) MECHANISM AND TECHNOLOGY FOR INHIBITING METHANE PRODUCTION IN RUMINANTS

(71) Applicant: FENG SHENG YUAN, KEELUNG CITY (TW)

(72) Inventor: FENG SHENG YUAN, KEELUNG CITY (TW)

(21) Appl. No.: 17/851,477

(22) Filed: Jun. 28, 2022

(30) Foreign Application Priority Data
Mar. 2, 2022 (TW) 111107610

(51) Int. Cl. A23K 50/10; A23K 20/20

(52) U.S. Cl. CPC A23K 50/10; A23K 20/20

(57) ABSTRACT

A mechanism and technology for inhibiting methane production in ruminants, which mixes an oscillating magnet composition 1~3% into the feedstuff, so as to inhibit the generation and reproduction of methanogens in the gastrointestinal tract of ruminants, thereby reducing the production of methane; wherein, the composition weight percentage of the oscillating magnet composition includes 72%~82% a far-infrared natural mineral base material, 10%~20% a biochar, 2%~7% a seaweed element and an activated water agent composed of 0.4%~1% of a natural ore; wherein the far-infrared natural mineral base material emit far-infrared rays, make water molecules cluster smaller, thereby improving the dissolved oxygen in the gastrointestinal tract to inhibit of the generation and reproduction of methanogens.

### BACKGROUND OF THE INVENTION

### 1. Field of the Invention

The invention relates to a composition, especially the one that mixed into the feedstuff, it can emit far-infrared rays inside the body that make dissolved oxygen quickly enter the stomach, so as to inhibit the generation and reproduction of methanogens in the gastrointestinal tract of ruminants.

### 2. Description of the Related Art

In addition to carbon dioxide, the greenhouse gas that has the greatest impact on global warming is methane; methane (CH₄) is commonly known as biogas, and its ability to capture heat in the atmosphere is 25 times that of carbon dioxide; and according to the UNFCCC to promote carbon neutrality and zero discharge requirements, the reduction and control of greenhouse gases including methane, are bound to be subject to extremely strict attention and scrutiny.

Burping and exhausting (farting) of ruminants such as cattle and sheep is the main source of producing methane. To investigate the reason why ruminants produce a large amount of methane, as showing in FIG. 1, due to the large amount of organic matter, such as pastures and fodder fed to cattle and sheep, the organic matter is kept in an anaerobic (anoxic) environment for a long time in the gastrointestinal tract due to its rumination characteristics, lead to the mass production and reproduction of “methanogen”; when these “methanogens” metabolize and decompose organic matters, they will produce a large amount of methane by-products, and then discharge methane gas into the atmosphere in the way of burping or exhausting (farting) of cattle and sheep, as showing in FIG. 1; “methanogens” are strictly obligate anaerobic bacteria, which must survive and reproduce in an absolutely anaerobic state; As long as there is a small amount of dissolved oxygen (D.O.), it will have an inhibitory effect, and when the amount of dissolved oxygen is greater than 0.7 mg/L, the “methanogens” cannot survive; The growth and reproduction of “methanogens” is very slow, and “methanogens” can only be produced in a long-term anaerobic environment.

The gastrointestinal tract of ruminants is a closed space, with a large amount of organic matter and a wide variety of microbial strains. The strains can generally be roughly divided into three categories: aerobic bacteria, facultative bacteria and anaerobic bacteria. As for which type of bacteria Is the dominant strain, it is determined by the amount of dissolved oxygen in the gastrointestinal tract at that time, as showing in FIG. 3; In the initial state, there is still a certain amount of dissolved oxygen in the gastrointestinal tract, the dominant bacteria are aerobic bacteria, while the number of facultative bacteria and anaerobic bacteria is very small. The aerobic bacteria will metabolize a large amount of organic matter and do self-reproduction of aerobic bacteria, and consume a large amount of dissolved oxygen at the same time; when the external oxygen supply rate is not fast enough, and the rate of dissolved oxygen consumption is greater than the rate of supplementation, the dissolved oxygen in the stomach will decrease, and when it falls to a certain extent, facultative bacteria will become dominant bacteria, while only there are a small amount of aerobic bacteria and anaerobic bacteria, and the dissolved oxygen is continuously consumed;

When the dissolved oxygen in the gastrointestinal tract continues to drop to 0 mg/L, it will be in a completely anaerobic state, anaerobic bacteria will become dominant bacteria, while aerobic bacteria and facultative bacteria will hardly exist, at this time, inside the gastrointestinal tract of ruminants will produced large amounts of methane gas and will be released into the atmosphere in the form of burps or exhaust (farts).

Researchers have tried to find a method to reduce the emission of methane from ruminants, including changing the feed formula to promote gastrointestinal digestion, and destroying specific digestive enzymes to achieve the effect of inhibiting specific bacterial species; for example, add asparagopsis taxiformis or grape pomace with a certain proportion into the feedstuff; however, these additive cannot effectively increase the amount of dissolved oxygen in the gastrointestinal tract to inhibit the generation and reproduction of methanogens, so as to reducing the amount of methane produced by ruminants.

### SUMMARY OF THE INVENTION

It is a primary objective of the present invention to provide a mechanism and technology for inhibiting methane production in ruminants is to provide a feedstuff with far-infrared natural mineral base material for feeding ruminants to increase the amount of dissolved oxygen in the stomach to inhibit the generation of methanogens.

In order to achieve the above objectives, the present invention comprise: mixes the oscillating magnet composition 1~3% into the feedstuff, so as to inhibit the generation and reproduction of methanogens in the gastrointestinal tract of ruminants, thereby reducing the production of methane; wherein, the composition weight percentage of the oscillating magnet composition includes 72%~82% a far-infrared natural mineral base material, 10%~20% a biochar, 2%~7% a seaweed element and an activated water agent composed of 0.4%~1% of a natural ore; wherein the far-infrared natural mineral base material can continuously and stably emit far-infrared rays, make the water molecules cluster in the gastrointestinal tract, blood vessels, lymph become smaller, and the smaller water molecules are easier to penetrate the film and facilitate diffusion; the CO₂ metabolized by organic matter is adsorbed on the surface of the biochar to reduce the concentration of CO₂ in water molecules, thereby reducing the amount of methane produced by methanogens; the seaweed element promote gastrointestinal digestion and destroy specific digestive enzymes to achieve the effect of inhibiting specific bacterial species; the activated water agent composed of a natural ore, its main components contain trace elements including BaO, Cr₂O₃, CaCO₃, P₂O₅, Na₂O, MnO, CaO, Cu, etc., it is used for increasing the amount of dissolved oxygen in water molecules.

The activated water agent is composed of a natural ore, it has a porous honeycomb shape and has the function of increasing the amount of dissolved oxygen in the water. Its mechanism and principle are as follows:
a. The active water agent is a natural ore. After a high temperature process in nature, it will continue to emit far infrared rays to make the water molecules cluster smaller, which will facilitate the diffusion of oxygen into water and dissolved oxygen.
b. The appearance of the active water agent is in the shape of a porous honeycomb. When the water flows through, it has the functions of dispersing the water flow and breaking up the water flow, which will increase the surface area of the contact between oxygen and water molecules, thereby increasing the dissolved oxygen speed and dissolved oxygen amount.
c. The active water agent has a water purification mechanism, which can remove pollutants in water, organic nitrogen (nitrogen in the form of organic matter, such as urea) and ammonia nitrogen (nitrogen in the form of ammonia, including NH₄+ and NH₃); The first step is nitrification, which is oxidizing organic nitrogen and ammonia nitrogen into nitrate, and the second step is denitrification, which is reducing nitrate into completely harmless nitrogen (chemical equation is as follows):

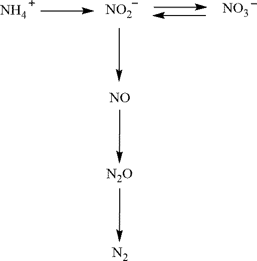

Since the active water agent is a natural ore, and the porous honeycomb shape makes its surface an excellent environment for “nitrifying bacteria” and “denitrifying bacteria” to attach and grow, the active water agent is conducive to removing organic nitrogen and ammonia nitrogen in water. Eutrophication can be avoided, and the amount of dissolved oxygen in the water can be increased at the same time.

Whereby mixes the oscillating magnet composition 1~3% into the feedstuff, the oscillating magnet composition continuously and stably emit far-infrared rays in the gastrointestinal tract make the water molecules inside resonate to make the water molecules cluster smaller, so as to make the dissolved oxygen in the blood penetrate more quickly into the film and diffuse in the gastrointestinal tract, thereby improving the dissolved oxygen in the gastrointestinal tract to achieves the inhibition of the generation and reproduction of methanogens, thereby let the ruminants to reduce methane gas production.

Also, the composition and weight percentage of the far-infrared natural mineral base material are: silicon dioxide (SiO₂) 44~53%, iron oxide (Fe₂O₃) 12~17%, manganese dioxide (MnO₂) 6~8%, calcium oxide (CaO) 3~6%, zirconium dioxide (ZrO₂) 3~7%, alumina (Al₂O₃) 6~15%, zinc oxide (ZnO) 2~4%, potassium oxide (K₂O) 3~5%, magnesium oxide (MgO) 1~3%, cobalt oxide (CoO) 2~4%, titanium dioxide (TiO₂) 1~3%, cerium oxide (CeO₂) 0.5-1%, and lanthanum oxide (La₂O₃) 0.1~0.5%; and make its far-infrared radiation rate reach the characteristic value of more than 88.8%.

With the features disclosed above the mechanism and technology for inhibiting methane production in ruminants mixes the oscillating magnet composition 1~3% into the feedstuff, the oscillating magnet composition continuously and stably emit far-infrared rays in the gastrointestinal tract make the water molecules inside the gastrointestinal tract blood vessels, lymph resonate to make the water molecules cluster smaller, so as to make the water molecules cluster penetrate easily through the microscopic holes in the walls of blood vessels and the membranes of the gastrointestinal tract; moreover, ruminants inhale air into the lungs, and oxygen dissolves in the water molecules in the blood to form dissolved oxygen (D.O.). When the blood circulates in the body, the dissolved oxygen (D.O.) in the water molecules is transported to various organs through the lymphatic system, so the miniaturization of water molecules allows dissolved oxygen in the blood to penetrate the membrane more quickly and diffuse into the gastrointestinal tract, thereby improving the dissolved oxygen in the gastrointestinal tract to achieves the inhibition of the generation and reproduction of methanogens, thereby let the ruminants to reduce methane gas production.

### BRIEF DESCRIPTION OF THE DRAWINGS

FIG. 1 is a block diagram illustrating the reproduction of the methanogens;

FIG. 2 is a schematic diagram illustrating ruminants emit methane through burping or exhausting;

FIG. 3 is a graph illustrating the proportion of bacteria in the gastrointestinal tract of ruminants and the amount of dissolved oxygen;

FIG. 4 is a block diagram illustrating the composition of the oscillating magnet of the present invention;

FIG. 5A is a schematic diagram illustrating the covalent bond structure between hydrogen and oxygen atoms;

FIG. 5B is a schematic diagram illustrating the water molecule polarity;

FIG. 5C is a schematic diagram illustrating the water molecule polarity;

FIG. 5D is a schematic diagram illustrating the water molecules cluster;

FIG. 5E is a schematic diagram illustrating the water molecules cluster go through the hole of the film;

FIG. 5F is a schematic diagram illustrating the circulation of blood in lymph system;

FIG. 6A is a schematic diagram illustrating the photomicrographs of the far-infrared natural mineral base material;

FIG. 6B is a schematic diagram illustrating the photomicrographs of the far-infrared natural mineral base material;

FIG. 6C is a schematic diagram illustrating the photomicrographs of the far-infrared natural mineral base material.

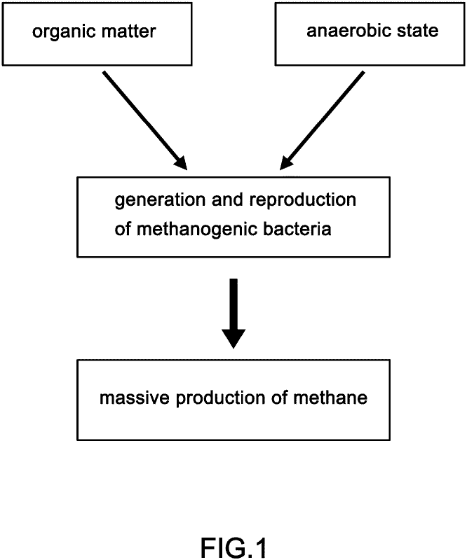

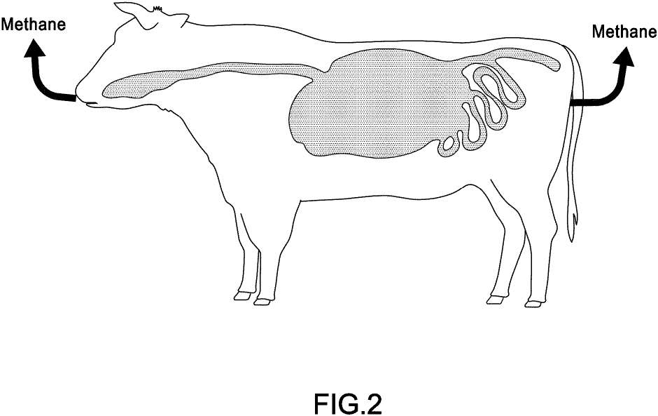

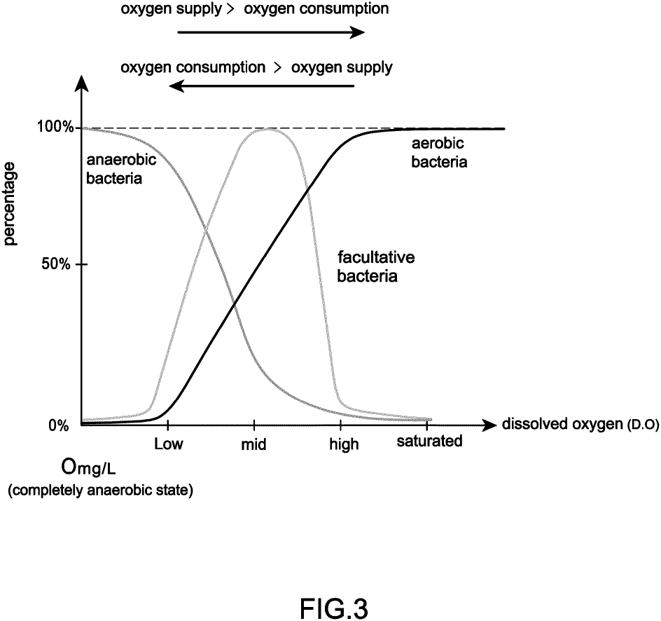

### DETAILED DESCRIPTION OF THE PREFERRED EMBODIMENT

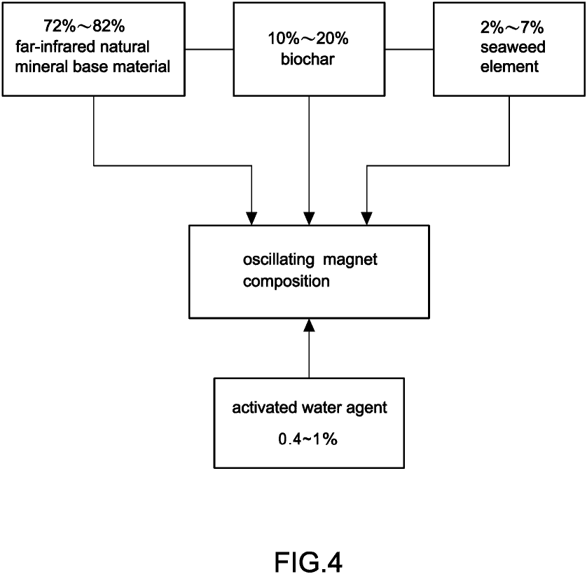

First, the present invention comprise: mixes the oscillating magnet composition 1~3% into the feedstuff, so as to inhibit the generation and reproduction of methanogens in the gastrointestinal tract of ruminants, thereby reducing the production of methane; wherein, as FIG. 4 showing, the composition weight percentage of the oscillating magnet composition includes 72%~82% a far-infrared natural mineral base material, 10%~20% a biochar, 2%~7% a seaweed element and an activated water agent composed of 0.4%~1% of a natural ore; wherein the far-infrared natural mineral base material can continuously and stably emit far-infrared rays, make the water molecules cluster in the gastrointestinal tract, blood vessels, lymph become smaller, and the smaller water molecules are easier to penetrate the film and facilitate diffusion; the CO₂ metabolized by organic matter is adsorbed on the surface of the biochar to reduce the concentration of CO₂ in water molecules, thereby reducing the amount of methane produced by methanogens; the seaweed element promote gastrointestinal digestion and destroy specific digestive enzymes to achieve the effect of inhibiting specific bacterial species; the activated water agent composed of a natural ore, its main components contain trace elements including BaO, Cr₂O₃, CaCO₃, P₂O₅, Na₂O, MnO, CaO, Cu, etc., it is used for increasing the amount of dissolved oxygen in water molecules.

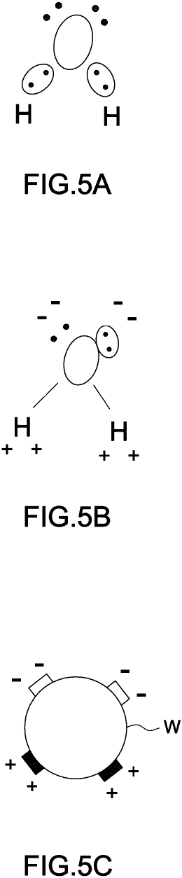

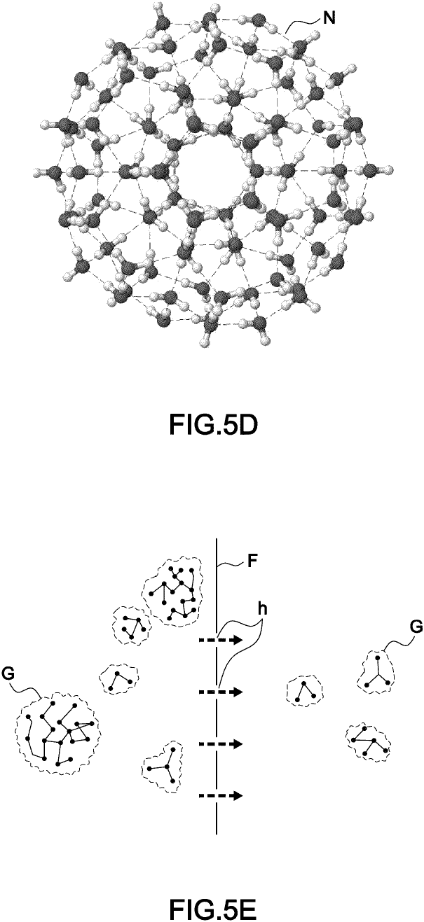

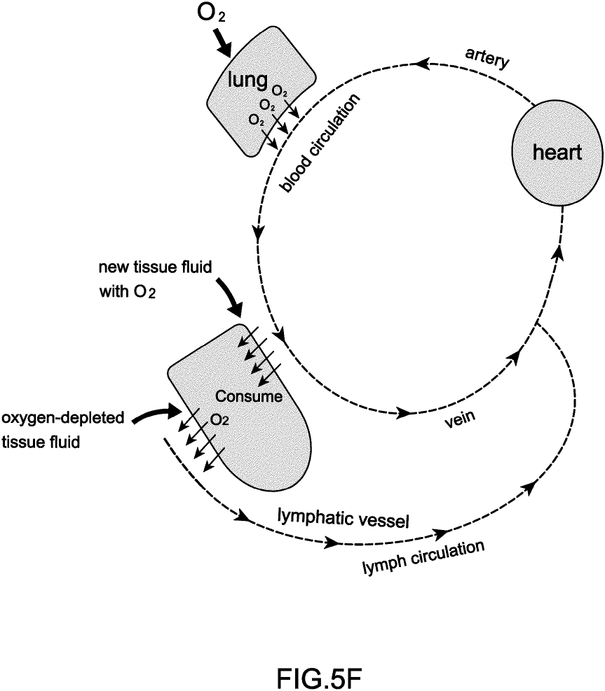

The far-infrared natural mineral base material of the present invention can continuously and stably emit far-infrared rays, make the water molecules cluster in the soil resonate to make the water molecules cluster smaller, and allows dissolved oxygen in the blood to penetrate the membrane more quickly and diffuse into the gastrointestinal tract, thereby improving the dissolved oxygen in the gastrointestinal tract; the mechanism and principle are as follows: a water molecule is composed of one oxygen atom and two hydrogen atoms, and its chemical formula is H₂O; wherein, one oxygen atom has 8 electrons, including 2 electrons in the first orbital, and 6 electrons in the second orbital, and the outer orbital (the second orbital) must have 8 electrons to maintain its stable structure; a hydrogen atom only has 1 electron in its first orbital, and the outer orbital (the first layer) must have 2 electrons to maintain stability; therefore, between one oxygen atom and two hydrogen atoms, it is stabilized in the form of covalent bonds shared by electrons, and water molecules are formed accordingly, as showing in FIG. 5A; in addition to covalent bonds in water molecules, the unshared electron pair at the oxygen atom end will be negative electrification, while the hydrogen atom end will be positive electrification, as showing in FIG. 5B; the polarity of water molecules W is like making four magnets with different polarities attached to the four opposite corners of a ping pong ball, wherein, two are positive hydrogen atoms, and the other two are negative unshared electron pairs, as showing in FIG. 5C; in liquid water, water molecules attract each other like ping-pong balls attached to magnets and form cluster, this attraction is called hydrogen bond, and promotes the phenomenon that liquid water forms water molecules cluster, that is, from a few to several cluster, ten or even hundreds of water molecules form a large reticular structure N, as showing in FIG. 5D.

The blood vessel wall and the gastrointestinal tract in ruminants have a film F full of fine holes h, the water molecules cluster G must pass through the fine holes h of the film F to enter the blood vessel wall and the gastrointestinal tract, and the smaller the water molecule cluster G, the easier it is for dissolved oxygen to pass through the film F, and the more the dissolved oxygen can diffuse into the blood vessel wall and the gastrointestinal tract, as showing in FIG. 5E; the far-infrared natural mineral base material developed can emit far-infrared rays with nano-wave energy, and its frequency can make the water molecules resonate, and the hydrogen bonds are broken by the vibration to decompose the water molecules to achieve the miniaturization of water molecules and then spreads into the gastrointestinal tract; in addition, ruminants inhale air into the lungs, and the oxygen dissolves in the water molecules in the blood to form dissolved oxygen (D.O.), when the blood circulates in the body, the dissolved oxygen (D.O.) in the water molecules cluster is transported to various organs and tissues through the lymphatic system, as showing in FIG. 5F; therefore, the miniaturization of the water molecule cluster can allow the dissolved oxygen in the blood to penetrate the film more quickly and diffuse into the gastrointestinal tract, thereby increasing the amount of dissolved oxygen in the gastrointestinal tract, and further inhibit the generation and reproduction of methanogens, so that ruminants can reduce the production of methane gas.

The composition and weight percentage of the far-infrared natural mineral base material of the present invention are: silicon dioxide (SiO₂) 44 ~53%, iron oxide (Fe₂O₃) 12~17%, manganese dioxide (MnO₂) 6~8%, calcium oxide (CaO) 3-6%, zirconium dioxide (ZrO₂) 3~7%, alumina (Al₂O₃) 6~15%, zinc oxide (ZnO) 2~4%, potassium oxide (K₂O) 3~5%, magnesium oxide (MgO) 1~3%, cobalt oxide (CoO) 2~4%, titanium dioxide (TiO₂) 1~3%, cerium oxide (CeO₂) 0.5~1%, and lanthanum oxide (La₂O₃) 0.1-0.5%; and make its far-infrared radiation rate reach the characteristic value of more than 88.8%.

According to the experimental method in the present invention, the composition of the far-infrared natural mineral base material is combined into 10 groups of base materials of different proportions according to different weight percentages, and its far-infrared radiation rate is detected respectively, the percentage of each group composition and the data of its radiation rate are showing in Table below:

| Group | 1 | 2 | 3 | 4 | 5 | 6 | 7 | 8 | 9 | 10 |
|---|---|---|---|---|---|---|---|---|---|---|
| Composition | | | | | | | | | | |
| SiO₂ | 44 | 43 | 44 | 45 | 47 | 48 | 50 | 49 | 51 | 53 |
| Fe₂O₃ | 16.5 | 17 | 13 | 14 | 14 | 15 | 14.8 | 14 | 13 | 12.4 |
| MnO₂ | 6 | 6 | 6.5 | 6.5 | 7 | 7 | 7 | 8 | 7 | 7 |
| CaO | 5 | 4 | 6 | 5 | 5 | 3 | 3 | 4 | 4 | 3 |
| ZrO₂ | 3 | 3 | 3.5 | 3.5 | 4 | 5 | 5 | 5 | 6 | 6.5 |
| Al₂O₃ | 12.5 | 14.5 | 11.5 | 10.5 | 7.5 | 8.5 | 7.5 | 6.7 | 6.2 | 6.5 |
| ZnO | 3 | 2 | 3.5 | 3 | 3.5 | 3 | 3 | 2.5 | 2 | 2 |
| K₂O | 3 | 3.5 | 5 | 4.5 | 4 | 3 | 3 | 3.5 | 4 | 4 |
| MgO | 3 | 3 | 2.5 | 2.5 | 2 | 1.5 | 1 | 1 | 1 | 1 |
| CoO | 2 | 2 | 2 | 2.5 | 2.5 | 3 | 3.5 | 4 | 3 | 3 |
| TiO₂ | 1 | 1 | 1.5 | 2 | 2.5 | 2 | 1.5 | 1.5 | 2 | 1 |
| CeO₂ | 0.7 | 0.7 | 0.7 | 0.5 | 0.6 | 0.8 | 0.6 | 0.7 | 0.5 | 0.5 |
| La₂O₃ | 0.3 | 0.3 | 0.3 | 0.5 | 0.4 | 0.2 | 0.1 | 0.1 | 0.3 | 0.1 |
| radiation rate | 88.8 | 89.6 | 90.4 | 91.1 | 92 | 95.1 | 96.3 | 95.3 | 92.4 | 89.3 |

Through the comparison of the far-infrared radiation rate of each group, it can be known that the three groups with the best radiation rate are Group 6, Group 7, and Group 8; wherein the composition percentages and the radiation rate for Group 7 are: silicon dioxide (SiO₂) 50%, iron oxide (Fe₂O₃) 14.8%, manganese dioxide (MnO₂) 7%, calcium oxide (CaO) 3%, zirconium dioxide (ZrO₂) 6%, alumina (Al₂O₃) 7.5%, zinc oxide (ZnO) 3%, potassium oxide (K₂O) 3%, magnesium oxide (MgO) 1%, cobalt oxide (CoO) 3.5%, titanium dioxide (TiO₂) 1.5%, cerium oxide (CeO₂) 0.6 %, and lanthanum oxide (La₂O₃) 0.1%, and radiation rate 96.3%; the composition percentages and the radiation rate for Group 8 are: silicon dioxide (SiO₂) 49%, iron oxide (Fe₂O₃) 14%, manganese dioxide (MnO₂) 8%, calcium oxide (CaO) 4%, zirconium dioxide (ZrO₂) 5%, alumina (Al₂O₃) 6.7%, zinc oxide (ZnO) 2.5%, potassium oxide (K₂O) 3.5%, magnesium oxide (MgO) 1%, cobalt oxide (CoO) 1.4%, titanium dioxide (TiO₂) 1.5%, cerium oxide (CeO₂) 0.7%, and lanthanum oxide (La₂O₃) 0.1%, and radiation rate 95.3%; the composition percentages and the radiation rate for Group 6 are: silicon dioxide (SiO₂) 48%, iron oxide (Fe₂O₃) 15%, manganese dioxide (MnO₂) 7%, calcium oxide (CaO) 3%, zirconium dioxide (ZrO₂) 5%, alumina (Al₂O₃) 8.5%, zinc oxide (ZnO) 3%, potassium oxide (K₂O) 3%, magnesium oxide (MgO) 1.5%, cobalt oxide (CoO) 3%, titanium dioxide (TiO₂) 2%, cerium oxide (CeO₂) 0.8%, and lanthanum oxide (La₂O₃) 0.2%, and radiation rate 95.1%.

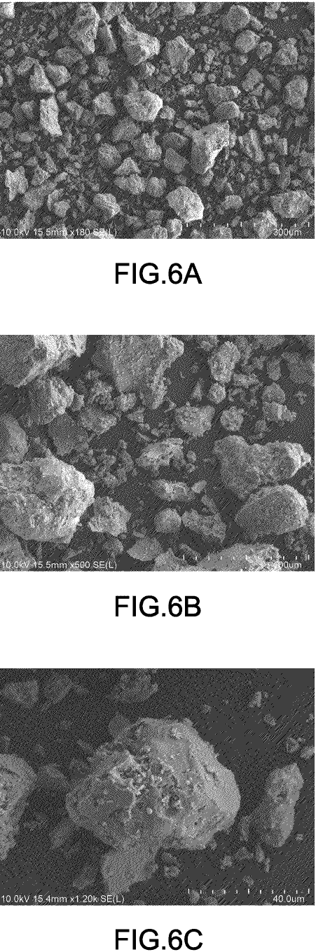

The far-infrared natural mineral base material in the present invention is a porous structure, and its pore diameter will reach the characteristic requirements of 0.2~0.8 microns; moreover, the porous structure formed by the far-infrared natural mineral base material is showing in FIG. 6A for a photo magnified by a microscope of 180 times, the magnification of 500 times is showing in FIG. 6B, the magnification of 1200 times is showing in FIG. 6C, the pore structure showing in the photo has a pore size of 0.2 to 0.8 microns, and emit far-infrared rays with a wavelength of 8~14 microns with nano-wave energy, because its frequency can just make water molecules resonate, and the hydrogen bonds can be broken by resonation to decompose the water molecules, and effectively promote the diffusion of the miniaturized oxygen-dissolved water molecules cluster into the gastrointestinal tract.

The mechanism and technology for inhibiting methane production in ruminants mixes the oscillating magnet composition 1~3% into the feedstuff, the oscillating magnet composition continuously and stably emit far-infrared rays in the gastrointestinal tract make the water molecules inside the gastrointestinal tract blood vessels, lymph resonate to make the water molecules cluster smaller, so as to make the water molecules cluster penetrate easily through the microscopic holes in the walls of blood vessels and the membranes of the gastrointestinal tract; moreover, ruminants inhale air into the lungs, and oxygen dissolves in the water molecules in the blood to form dissolved oxygen (D.O.). When the blood circulates in the body, the dissolved oxygen (D.O.) in the water molecules is transported to various organs through the lymphatic system, so the miniaturization of water molecules allows dissolved oxygen in the blood to penetrate the membrane more quickly and diffuse into the gastrointestinal tract, thereby improving the dissolved oxygen in the gastrointestinal tract to achieves the inhibition of the generation and reproduction of methanogens, thereby let the ruminants to reduce methane gas production.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A mechanism and technology for inhibiting methane production in ruminants, which mixes an oscillating magnet composition 1~3% into the feedstuff, so as to inhibit the generation and reproduction of methanogens in the gastrointestinal tract of ruminants, thereby reducing the production of methane; wherein, the composition weight percentage of the oscillating magnet composition includes 72%~82% a far-infrared natural mineral base material, 10%~20% a biochar, 2%~7% a seaweed element and an activated water agent composed of 0.4%~1% of a natural ore.

2. The mechanism and technology for inhibiting methane production in ruminants as claimed in claim 1, wherein the activated water agent composed of a natural ore, its main components contain trace elements including BaO, Cr₂O₃, CaCO₃, P₂O₅, Na₂O, MnO, CaO, Cu, etc.

3. The mechanism and technology for inhibiting methane production in ruminants as claimed in claim 1, wherein the composition and weight percentage of the far-infrared natural mineral base material are: silicon dioxide (SiO₂) 44~53%, iron oxide (Fe₂O₃) 12~17%, manganese dioxide (MnO₂) 6~8%, calcium oxide (CaO) 3~6%, zirconium dioxide (ZrO₂) 3~7%, alumina (Al₂O₃) 6~15%, zinc oxide (ZnO) 2~4%, potassium oxide (K₂O) 3~5%, magnesium oxide (MgO) 1~3%, cobalt oxide (CoO) 2~4%, titanium dioxide (TiO₂) 1~3%, cerium oxide (CeO₂) 0.5~1%, and lanthanum oxide (La₂O₃) 0.1~0.5%; and make its far-infrared radiation rate reach the characteristic value of more than 88.8%.
